(19) United States
(12) Patent Application Publication
(10) Pub. No.: US 2026/0258372 A1
(43) Pub. Date: Sep. 3, 2026

(54) Methods and Compositions for the Enrichment of Nucleic Acid Containing AAV Capsids

(71) Applicant: Brammer Bio, LLC, Cambridge, MA (US)

(72) Inventor: Hetal Brahmbhatt, St Augustine, FL (US)

(21) Appl. No.: 18/881,698

(22) PCT Filed: Jul. 11, 2023
(86) PCT No.: PCT/US2023/027382

§ 371 Date: Jan. 6, 2025

Related U.S. Application Data
(60) Provisional application No. 63/388,583, filed on Jul. 12, 2022.

(51) Int. Cl. C12N 7/00; B01D 15/16; B01D 15/36

(52) U.S. Cl. CPC C12N 7/00; B01D 15/166; B01D 15/363; C12N 2750/14151

(57) ABSTRACT

The present disclosure exemplifies methods and compositions by which full recombinant adeno-associated virus (AAV) capsids are enriched from a composition having both empty and full AAV capsids. Demonstrated herein is that full AAV capsids can be enriched using an isocratic step gradient wherein the elution buffers are pre-made and do not, therefore, require on-line formulation. This advancement improves the ease of automation at manufacturing scale by reducing operational errors or failures and in turn lost AAV therapeutic batches.

### RELATED APPLICATIONS

This application is a U.S. 371 National Phase application off of International Application Number PCT/US2023/027382, filed Jul. 11, 2023, which claims the benefit of U.S. Provisional Application No. 63/388,583 filed on Jul. 12, 2022.

### BACKGROUND

Methods for purifying adeno-associated virus particles include size-exclusion and affinity chromatography using antibodies specific for AAV capsid proteins. Affinity chromatography, although highly selective for the AAV capsid, cannot discriminate between a full (genome-containing) viral particle and an empty viral particle. Increased levels of empty viral particles within a AAV preparation reduces therapeutic efficacy and could enhance host immune responses.

Various methods for separating empty and full AAV particles in order to enrich viral compositions for full AAV viral particles have been proposed. Empty AAV viral particles can be effectively separated from full AAV particles by density gradient centrifugation using cesium chloride or iodixanol gradients based on the differing densities between the full (1.40 g/cm³) and empty (1.32 g/cm³) viral particles. However, these methods are not easily scalable and are costly.

Other proposed methods for separating empty and full AAV particles take advantage of the subtle difference in surface charge between a full AAV particle (average pi of about 5.9) and empty AAV particle (average pi of about 6.3) by using ion exchange chromatography (IEX). Although the separation of AAV empty and full particles using ion exchange chromatography with various media for cation or anion exchange has been reported, generally, the process is not easily optimized and requires significant experimentation in which success may not be guaranteed.

Thus, there remains a need for scalable methods for purifying full AAV particles that can be used for the enrichment of full viral particles of multiple AAV serotypes.

### BRIEF SUMMARY

The development of recombinant adeno-associated virus (AAV) gene therapies is becoming an increasing priority in the bio-therapeutic landscape. A challenge associated with the production of AAV is the formation of AAV capsids that do not contain a therapeutic gene. The concerns about the impact of such particles on clinical safety and AAV-mediated gene expression necessitated development of purification processes to remove these species. The development of a robust and scalable purification process to enrich for AAV capsids that contain a therapeutic gene at large scale remains challenging. Herein disclosed are methods and compositions that address these challenges.

In some embodiments, a method for enriching for full AAV capsids is disclosed, the method encompassing loading a sample suspected of having empty and full AAV capsids onto an anion exchange chromatography medium, differentially eluting the full AAV capsids and the empty AAV capsids with an elution buffer, wherein the composition of the elution buffer is not prepared in-line.

In other embodiments, a method for identifying an elution buffer for enriching for full AAV capsids is disclosed, wherein the elution buffer formulation not being prepared in-line, the formulation being arrived at by performing a linear gradient and an isocratic step anion exchange chromatography, and through the combined results arriving at an elution buffer formulation.

An elution buffer composition for enriching for full AAV capsids, wherein the elution buffer is not formulated in-line.

### DESCRIPTION OF THE DRAWINGS

FIG. 1. Schematic representation of using predetermined isocratic buffer conditions in AAV purification at manufacturing scale. The starting material is eluent from an AAV affinity resin (“AAVX Eluate”). The AAVX Eluate undergoes a load adjustment step that alters the pH and conductivity or salt concentration (“AEX load”). The AEX load is applied to an anion exchange media. Elution is conducted using an elution buffer that is not prepared in-line.

FIG. 2. Linear Gradient Scouting Run—Lower Conductivity Chromatogram.

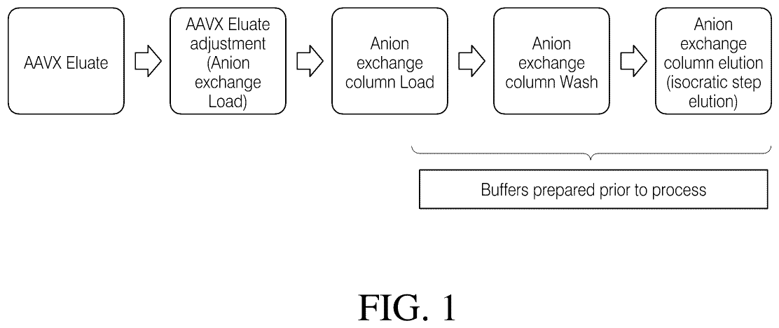

Chromatograph from the anion exchange elution step of rAAV9.GFP. Conductivity values at various points during the gradient are indicated as black circles with values in mS/cm. Two crossover points are seen at ~1.0 and 3.5 mS/cm. Fractions were pooled based on the following criteria E1: Region neighboring crossover, E2: Peak between two crossover points, E3: Peak after crossover point, E4: Peak region. E3 had a yield of 71% transgene containing particles enriched by a factor of 1.9 with ≤5% product loss in the flow through fraction. Chromatogram shows that starting of the first peak and including the fraction containing the most transgene containing particles is ~1 to ~8 mS/cm. Based on the decision outlined in Table 2, the AAVX Eluate is adjusted to a target conductivity of 1.5-2.5 mS/cm.

FIG. 3. Linear Gradient Scouting Run—Higher Conductivity Chromatogram. Chromatograph from the anion exchange elution step of rAAV9.GFP. Conductivity values at various points during the gradient are indicated as black circles with values in mS/cm. Fractions were pooled based on the following criteria E1: Peak region, E2: Peak region. E1 had the highest yield of 9% of transgene containing particles enriched by a factor of 1.4. 64% of the transgene containing particles were present in the flow through fraction. Chromatogram shows that starting of the first peak and including the elution fraction containing the most transgene containing particles is ~3.6 to ~8 mS/cm. Based on the decision outlined in Table 2, the AAVX Eluate should be adjusted to a target conductivity of 1.5-2.5 mS/cm given that all other target criteria for Table 1/experiment #1 are also met.

FIG. 4. Linear Gradient Scouting Run—Higher Conductivity Chromatogram. Chromatograph from the anion exchange elution step of rAAV2.GFP. Conductivity values at various points during the gradient are indicated as black circles with values in mS/cm. Fractions were pooled based on the following criteria E1: Region between and nearing two crossover points, E2: Peak region, region after cross over, E3: Peak region. E2 had the highest yield of 43% of transgene containing particles enriched by a factor of 1.9. E1 contained the highest yield of transgene lacking particles with ≤5% product loss in the flow through. Chromatogram shows that starting of the first peak and including the fraction containing the most transgene containing particles is ~8 to ~14 mS/cm. Based on Table 2, the AAVX Eluate should be adjusted to a target conductivity of 3.5-4.5 mS/cm.

FIG. 5. Chromatograph from the anion exchange elution step of rAAV2.GFP. Conductivity values at various points during the isocratic step gradient elutions (increases by ~2 mS/cm) are indicated as black circles with values in mS/cm. Ranges for the conductivities were established from the range of ~8 to ~14 mS/cm seen in FIG. 4. Purification yields and enrichment of transgene containing particles is described in Table 3. Results indicate that enriched transgene containing particles elute at conductivity values of ~12 mS/cm. The crossover point using the linear gradient in FIG. 4 is at ~10 mS/cm and the E2 collected after this point also have the highest portion of enriched particles (FIG. 4). A higher portion of transgene lacking particles can be eluted when the conductivity is ~8-10 mS/cm. Based on Table 6, buffer containing 70 mM and/or 80 mM sodium chloride would eliminate product impurities while a buffer that has 90 mM and/or 100 mM sodium chloride should enrich transgene containing particles.

FIG. 6. High load conductivity scouting run. Chromatograph from the anion exchange elution step of rAAV6.GFP. Conductivity values at various points during the gradient are indicated as black circles with values in mS/cm. Fractions were pooled based on the following criteria: E1: Region neighboring crossover, E2: Peak after crossover. E2 contained the highest purification yield of transgene containing particles of 47% and had 1.6× transgene enriched particles as compared to E1. Chromatogram shows that the starting the first peak and including the most transgene containing particles is ≥10.5. Based on the decision outlined in Table 2 the AAVX Eluate is adjusted to a target conductivity of 1.5-2.5 mS/cm.

FIG. 7. Chromatograph from the anion exchange elution step of rAAV6.GFP. Conductivity values at various points during the isocratic gradient elutions are indicated as black circles with values in mS/cm. Elution 2 has a purification yield of 0.2% with an enrichment of 0.008× whereas Elution 3 has a purification yield of 61% with an enrichment of 1.36× relative to the load material. Based on these results transgene lacking particles can be eluted at ~13.8 mS/cm whereas transgene enriched particles can be eluted at ≥13.8 to ~19.2 mS/cm. Based on Table 5, buffer containing 100-120 mM sodium chloride would eliminate product impurities while a buffer containing 140-180 mM sodium chloride should enrich transgene containing particles.

### DETAILED DESCRIPTION

Gene therapy has evolved over the past decade into a promising therapeutic class for treating many intractable diseases. Adeno-associated virus (AAV) is one of the most commonly used viral vector for delivering therapeutic genes.

An inherent characteristic of the AAV manufacturing process is the production of capsids that are not packaged with a nucleic acid. Gene therapy programs have shown positive correlation generally between the presence of gene copy numbers (vg/kg patient weight) and intended therapeutic benefit. Capsids devoid of nucleic acids are unable to provide a therapeutic benefit and can impair potency through receptor competition.

Separation of capsids containing a nucleic acid from those without at preparative scale is often accomplished by anion exchange chromatography (AEX). This is because AEX is scalable and capable of yielding high-titer AAV stocks. Unfortunately, capsids with and without a nucleic acid are of similar charge and size. This makes separation of capsids containing a nucleic acid from those without extremely challenging, often relying on operator judgment or on non-robust automation approaches.

The long-standing paradigm of those in the field, until the present disclosure, was that in-line preparation and adjustment of elution buffers using linear or step gradients during the AEX process was required to ensure separation of capsids containing a nucleic acid from those without. Intervention in elution buffer formulation, through controlled in-line buffer mixing, or elution collection criteria, is often necessary to meet the tolerances required to distinguish capsids with so close physical properties. It is now shown herein that capsids containing a nucleic acid from those without can be separated without resorting to in-line elution buffer formulation. This paradigm shifting methodology, and associated compositions, will reduce failures during the AAV isolation process resulting in saved time, money, and most importantly, the delivery of necessary therapeutics.

Accordingly, herein disclosed, are methods for enriching full from empty AAV capsids, the methods encompassing loading a sample suspected of having full and empty AAV capsids onto an anion exchange chromatography medium, differentially eluting the full from empty AAV capsids with an elution buffer, the composition of the elution buffer not being prepared in-line. “In-line” referring to the flow pathway of an appropriate liquid, such as a buffer, from a liquid reservoir to the inlet of an anion exchange chromatography medium.

A “full” AAV capsid refers to an outer shell of AAV proteins, VP1, VP2, and/or VP3, encapsulating a polynucleotide. An “empty” AAV capsid refers to an outer shell of AAV proteins, VP1, VP2, and/or VP3 that lacks an encapsulated polynucleotide. For gene therapy, the polynucleotide is a combination of nucleic acid sequences that do and do not occur within the AAV genome; a eukaryotic gene flanked by the inverted terminal repeat (ITR) sequence of AAV, for instance. In some embodiments, a capsid is full if a therapeutic polynucleotide is encapsulated. In still other embodiments, a capsid is full if two ITRs are encapsulated. In some embodiments, a capsid is full if a therapeutic polynucleotide and an ITR is encapsulated.

AAV capsid proteins have a greater UV absorbance at 280 nm than at 260 nm and the nucleic acid has greater UV absorbance at 260 nm than at 280 nm. As empty AAV particles lack a nucleic acid, they generally have a lower UV absorbance at 260 nm (A260) than full AAV particles, which include a nucleic acid. As a result, the A260/A280 ratio for an elution fraction that is enriched in empty AAV particles will be less than the A260/A280 ratio for an elution fraction that is relatively enriched in full AAV particles generally.

An elution fraction is recognized as enriched when it is estimated there are 1.1, 1.2, 1.3, 1.4, 1.5, 1.6, 1.7, 1.8, 1.9, 2.0, 2.5, 3, 5, 10, 20, 50, 100, or more times the number of full particles in an elution fraction relative to another or to the original sample; such as cell lysate, culture media supernatant, or affinity eluate. Alternatively, the percent of full capsids in a fraction relative to other fraction(s) can be used to measure enrichment. An elution fraction that is made up of 30%, 40%, 50%, 60%, 70%, 80%, 90% or more of full capsids while the original sample has a lower relative percentage of full capsids is an enriched elution fraction. An elution fraction can be less than, equal to, or greater than the chromatography medium volume.

Numerous serologically distinct AAV capsids are known. Currently, there are 12 naturally occurring AAV serotypes, denoted AAV1-12. AAV10 is alternatively referred to as AAVrh10. In addition to these naturally occurring serotypes, non-naturally occurring serotypes can be generated by genetic engineering. By splicing the genes encoding VP1, VP2, and/or VP3 of one serotype with those of another serotype a non-naturally occurring serotype can result. In some embodiments, the capsid is naturally occurring serotype. In other embodiments, the capsid is a non-naturally occurring serotype. In still other embodiments, the naturally occurring serotype is 1, 2, 3, 4, 5, 6, 7, 8, 9, rh10, 11, or 12.

At-scale AAV manufacturing can be divided into two general phases, an upstream phase where AAV is expanded by cell culture and a downstream phase in which the generated AAV is purified. During the upstream phase AAV, and depending on cell type, AAV can be collected from the culture supernatant, cell lysate, or both culture supernatant and cell lysate. The harvested material then enters the downstream phase. In the downstream phase the harvested material can be subjected to filtration, chromatography, including size exclusion, affinity and ion exchange chromatography, and ultracentrifugation. Often the downstream phase will be a combination of two or more of these techniques. For instance, the harvested material is subjected to filtration and then affinity chromatography and then ion exchange chromatography. Ion exchange chromatography can be either anion or cation, based on the conditions. The number and order of the techniques applied is not set and is often arrived at through experimentation. The material resulting from the application of a preceding technique is used as starting material for a subsequent technique, when more than one technique is applied.

At-scale manufacturing is when the culture medium harvest is at least 10 liters, 20 liters, 50 liters, 100 liters, 200 liters or more. These larger volumes distinguish at-scale manufacturing levels from research and small preparative scale, as is conducted in academic laboratories for example.

Accordingly, in some embodiments the sample can be a cell lysate. In other embodiments, the sample is a culture supernatant. In still other embodiments, the sample is both a cell lysate and a culture supernatant. In some embodiments, the sample is an eluent from an affinity column. In other embodiments, the sample is an eluent from an ion exchange column. In still other embodiments, the sample is a filtrate. In some embodiments, the sample is a band or pellet formed after centrifugation.

Therefore, there are methods disclosed herein for enriching full from empty AAV capsids, the full capsid being one with a therapeutic polynucleotide encapsulated, the sample suspected of having full and empty AAV capsids being the eluent of an affinity column, the eluent being loaded onto an anion exchange chromatography medium, differentially eluting the full from empty AAV capsids with an elution buffer, the composition of the elution buffer not being prepared in-line.

### Anion-Exchange Chromatography Medium

Anion exchange chromatography is a type of ion exchange chromatography in which ions are separated based on their ionic strength. The mobile phase is passed over the stationary phase, allowing negatively charged molecules from the mobile phase to bind to the positively charged anion exchange chromatography medium.

The methods of the present disclosure are not limited to any particular column structure or type of separation media. In some embodiments, the anion exchange chromatography medium can be housed within a closed environment, such as a column. In some embodiments, the column can be a monolith of anion exchange media. In other embodiments, the column may include packed particles of anion exchange media. In still other embodiments, a separation medium may be an anion exchange membrane.

Exemplary anion exchange chromatography medium include CIMMULTUS QA (available from BIA Separations, Ajdovscina, Slovenia), CIMMULTUS DEAE (available from BIA Separations, Ajdovscina, Slovenia), MACRO PREP Q (available from BioRad, Hercules, CA), MACRO PREP DEAE (available from BioRad, Hercules, CA), UNOSPHERE Q (available from BioRad, Hercules, CA), NUVIA Q (available from BioRad, Hercules, CA), POROS 50HQ (available from Thermo Fisher Scientific, Waltham, MA), POROS 50XQ (available from Thermo Fisher Scientific, Waltham, MA), POROS 50D (available from Thermo Fisher Scientific, Waltham, MA), POROS 50PI (available from Thermo Fisher Scientific, Waltham, MA), SOURCE 30Q (available from GE Healthcare, Uppsala, Sweden), MACROCAP Q (available from GE Healthcare, Uppsala, Sweden), DEAE SEPHAROSE FAST FLOW (available from GE Healthcare, Uppsala, Sweden), Q SEPHAROSE FAST FLOW (available from GE Healthcare, Uppsala, Sweden), Q SEPHAROSE HIGH PERFORMANCE (available from GE Healthcare, Uppsala, Sweden), CAPTO Q (available from GE Healthcare, Uppsala, Sweden), CAPTO Q IMPRES (available from GE Healthcare, Uppsala, Sweden), CAPTO DEAE (available from GE Healthcare, Uppsala, Sweden), Q SEPHAROSE (available from GE Healthcare, Uppsala, Sweden), Q SEPHAROSE XL (available from GE Healthcare, Uppsala, Sweden), STREAMLINE Q XL (available from GE Healthcare, Uppsala, Sweden), STREAMLINE DEAE (available from GE Healthcare, Uppsala, Sweden), ANX SEPHAROSE 4 FAST FLOW (available from GE Healthcare, Uppsala, Sweden), ESHMUNO Q (available from MilliporeSigma, Billerica, MA), FRACTOGEL TMAE (available from MilliporeSigma, Billerica, MA), FRACTOGEL DEAE (available from MilliporeSigma, Billerica, MA), CELLUFINE MAX Q (available from JNC corporation, Tokyo, Japan), CELLUFINE MAX DEAE (available from JNC corporation, Tokyo, Japan), Q CERAMIC HYPERD F (available from Pall Corporation, Westborough, MA), DEAE CERAMIC HYPERD F (available from Pall Corporation, Westborough, MA), SARTOBIND Q (available from Sartorius Stedim Biotech, Germany), SARTOBIND STIC (available from Sartorius Stedim Biotech, Germany), MUSTANG® Q (available from Pall Corporation, Westborough, MA), and NATRIFLO HD-Q (available from Burlington, Ontario, Canada).

In some embodiments, the anion exchange chromatography medium is MUSTANG® Q (available from Pall Corporation, Westborough, MA).

### AEX Scouting Runs

The instant disclosure represents a paradigm shift in the field of preparative AAV production, in part, because the composition of the elution buffer(s) applied to the anion chromatography system are unaltered during the journey through the flow path from a buffer reservoir to the anion exchange chromatography medium. There is no in-line combination of buffers of different compositions to arrive at further composition that is then applied to the anion exchange chromatography medium. This feat is accomplished through a series of linear gradient and step isocratic gradient AEX scouting experiments, described in detail in the Examples, ultimately identifying appropriate elution buffer formulations that are not prepared in-line.

Accordingly, in some embodiments a method for identifying an elution buffer for enriching full from empty AAV capsids is disclosed, the elution buffer formulation is not prepared in-line. The method encompassing loading a sample suspected of having full and empty AAV capsids onto an anion exchange chromatography medium, applying to the anion exchange chromatography medium a salt solution and collecting a series of elution fractions from anion exchange chromatography medium, wherein the concentration of salt is linearly increased during the gradient elution.

In some embodiments, the salt solution contains a monovalent salt. In other embodiments, the salt solution contains a divalent salt. In still other embodiments, the salt solution contains a monovalent salt and a divalent salt. In some embodiments, the monovalent salt is selected from sodium chloride, lithium chloride, ammonium chloride, sodium acetate and potassium chloride. In other embodiments, the monovalent salt is sodium chloride. In some embodiments, the divalent salt is selected from sodium monophosphate, sodium sulfate, zinc sulfate, zinc chloride, magnesium sulfate, calcium chloride and manganese chloride. In other embodiments, the divalent salt is sodium sulfate.

In some embodiments, the linear gradient is at an ionic strengthen of 0 mM to 1000 mM. In other embodiments, the linear gradient is at an ionic strengthen of 80 mM to 1000 mM. In still other embodiments, the linear gradient is at an ionic strengthen of 20 mM to 300 mM. In some embodiments, the linear gradient is at an ionic strengthen of 0 mM to 120 mM.

In some embodiments, a monovalent salt is the only salt. In still other embodiments, sodium chloride is the only monovalent salt. In some embodiments, the sodium chloride concentration during the linear gradient is at a concentration of 0 mM to 120 mM. In other embodiments, the sodium chloride concentration during the linear gradient is at a concentration of 20 mM to 300 mM. In still other embodiments, the sodium chloride concentration of 80 mM to 1000 mM.

In other embodiments, a divalent salt is the only salt. In still other embodiments the divalent salt is sodium sulfate. In some embodiments, the sodium sulfate during the linear gradient is at a concentration of 0 mM to 300 mM. In other embodiments, the sodium sulfate concentration during the linear gradient is at a concentration of 20 mM to 100 mM. In still other embodiments, the sodium sulfate concentration during the linear gradient is at a concentration of 20 mM to 30 mM.

Step isocratic AEX is also undertaken for identifying an elution buffer for enriching full from empty AAV capsids is disclosed, the elution buffer formulation not being prepared in-line.

Accordingly, in some embodiments a method for identifying an elution buffer formulation for enriching full from empty AAV capsids is disclosed, the formulation being arrived at through the combination of results from linear gradient and step isocratic anion exchange chromatography, the formulation of the linear gradient elution buffer being prepared in-line.

In some embodiments a method for identifying an elution buffer formulation for enriching full from empty AAV capsids is disclosed, the elution buffer formulation not being prepared in-line, the formulation being arrived at through the combination of results from a linear gradient and a step isocratic anion exchange chromatography, wherein the sample is an eluent from an affinity chromatography medium. To the sample is added an “AAVX eluate adjustment buffer” encompassing Bis-Tris Propane and Poly(ethylene glycol)-block-poly(propylene glycol)-block-poly(ethylene glycol). The sample plus AAVX eluate adjustment buffer is added to an anion exchange medium, followed by linear gradient or step isocratic chromatography. A representative overview of this process is provided in Table 1.

TABLE 1

| | Scouting Experiments | | | | Final Experiment |
|---|---|---|---|---|---|
| | Experiment | Experiment | Experiment | Experiment | Experiment |
| | #1 | #2 | #3 | #4 | #5 |
| AAVX Eluate | 1.5-2.5 | 3.5-4.5 | 10.5-11.5 | Determined from Experiment | |
| conductivity after | mS/cm | mS/cm | mS/cm | # 1 and/or #2 and/or 3 | |
| adjustment | | | | | |
| AAVX Eluate | pH > 8 | | | | |
| conductivity after | | | | | |
| adjustment | | | | | |
| Purification | Apply adjusted AAVX Eluate to anion exchange media | | | | |
| load step | | | | | |

| Purification | 10 mM Bis Tris Propane, 0.01% | Determined from | Determined from |
|---|---|---|---|
| wash Buffer | Kolliphor P-188 pH 9.0 including | Experiment # 1 | Experiment #4 |
| | 0-120 mM sodium chloride | and/or #2 and/or 3. | |
| | | Ionic strength of | |
| | | wash buffer is less | |
| | | than ionic strength | |
| | | of elution buffer | |
| Purification wash | In- line or not in-line | In- line or not in-line | Not in-line |
| buffer generation | | | |
| Elution step | Linear gradient | Step isocratic gradient | |
| method | | | |
| Elution wash | In- line | In- line or not in-line | Not in- line |
| buffer generation | | | |
| Elution | Linear gradient. | Target conductivity | Determined from |
| conditions | Buffer #1: 10 mM Bis Tris Propane, 0.01% Kolliphor | ranges or sodium | Experiment #4 |
| | P-188 pH 9.0 and sodium chloride from (0-1000 mM) | chloride | |
| | Buffer #2: 10 mM Bis Tris Propane, 0.01% Kolliphor | concentrations | |
| | P-188 pH 9.0 and sodium chloride from (0-1000 mM) | determined from | |
| | | Experiment #1 | |
| | | and/or #2 and/or | |
| | | Experiment #3. | |
| | | Increases in | |
| | | conductivity should | |
| | | be ≥1 mS/cm. | |
| Elution volume | Fraction volumes ≤2 column volumes | Volume ≥ column void volume | |
| Purification | Evaluate purification performance using vector genome analysis, | | |
| performance | capsid titer analysis and/or analytical ultracentrifugation and/ | | |
| evaluation | or SEC- MALS on select samples | | |
| Selection of | Fractions are selected based on the start and end | Determined from | Determined from |
| fractions for | regions of a peak and can include regions before, after | Experiment #1 | Experiment #4 |
| analysis | or within the peak region. A peak is defined as any | and/or #2 and/or | |
| | region wherein an increase and decrease in | Experiment #3 | |
| | absorbance (UV260 and/or UV280) is observed. | and #4. | |
| | Crossover points occur when UV260 and UV280 are of | | |
| | equal values. These may be observed at the start of a | | |
| | peak or at the end of a peak or between two peaks. | | |
| | Fractions neighboring crossover points may have | | |
| | UV280 greater than UV 260 values or UV260 values | | |
| | greater than UV260 values. Regions of interest can | | |
| | include fractions that have increasing or decreasing | | |
| | UV signal. | | |

The AEX scouting runs, both the linear gradient chromatography utilizes a mixer. A “Mixer” refers to a device that is deliberately used in a liquid chromatography system for the purpose of mixing two or more different liquids. For instance, mixing different ratios of two or more buffer solutions with low to high salt concentrations to deliver the desired salt concentration to the anion exchange chromatography medium.

Mixer use distinguishes the AEX scouting runs from the methods for enriching full from empty AAV capsids, the methods encompassing loading a sample suspected of having full and empty AAV capsids onto an anion exchange chromatography medium, differentially eluting the full from empty AAV capsids with an elution buffer, the composition of the elution buffer not being prepared in-line. Mixers formulate a buffer in-line for input into the anion exchange chromatography medium.

Thus, the methods disclosed herein encompass loading a sample suspected of having full and empty AAV capsids onto an anion exchange chromatography medium, differentially eluting the full from empty AAV capsids with an elution buffer, the elution buffer not resulting from in-line mixing of two or more buffers with a mixer. Many chromatography systems or “skids” possess mixers as an integral part, with the mixer occupying a position in the flow pathway from buffer reservoir to chromatography medium. While the elution buffer for enriching full from empty AAV capsids disclosed herein may pass through a mixer, the elution buffer that reaches the chromatography medium is not formulated at the mixer, through the combination of two or more buffers. From the AEX scouting runs the elution buffer composition(s) is identified.

### Elution Buffers

Through the use of AEX scouting runs, for instance, the combination of linear gradient and step isocratic gradient chromatography, elution buffer compositions were arrived at for use in enriching full from empty AAV capsids, the methods encompassing loading a sample suspected of having full and empty AAV capsids onto an anion exchange chromatography medium, differentially eluting the full from empty AAV capsids with an elution buffer, the composition of the elution buffer not being prepared in-line.

In some embodiments, the elution buffer contains a monovalent salt. In other embodiments, the elution buffer contains a divalent salt. In still other embodiments, the elution buffer contains a monovalent salt and a divalent salt. In some embodiments, the monovalent salt is selected from sodium chloride, lithium chloride, ammonium chloride, sodium acetate and potassium chloride. In other embodiments, the monovalent salt is sodium chloride. In some embodiments, the divalent salt is selected from sodium monophosphate, sodium sulfate, zinc sulfate, zinc chloride, magnesium sulfate, calcium chloride and manganese chloride. In other embodiments, the divalent salt is sodium sulfate.

In some embodiments, the elution buffer has an ionic strengthen of 0 mM to 1000 mM. In other embodiments, the elution buffer is at an ionic strengthen of 80 mM to 1000 mM. In still other embodiments, the elution buffer at an ionic strengthen of 20 mM to 300 mM. In some embodiments, the elution buffer is at an ionic strengthen of 0 mM to 120 mM.

In some embodiments, a monovalent salt is the only salt. In still other embodiments, sodium chloride is the only monovalent salt. In some embodiments, the sodium chloride in the elution buffer is at a concentration of 0 mM to 120 mM. In other embodiments, the sodium chloride concentration in the elution buffer is at a concentration of 20 mM to 300 mM. In still other embodiments, the sodium chloride in the elution buffer is at a concentration of 80 mM to 1000 mM.

In other embodiments, a divalent salt is the only salt. In still other embodiments the divalent salt is sodium sulfate. In some embodiments, the sodium sulfate in the elution buffer is at a concentration of 0 mM to 300 mM. In other embodiments, the sodium sulfate concentration in the elution buffer is at a concentration of 20 mM to 100 mM. In still other embodiments, the sodium sulfate concentration in the elution buffer is at a concentration of 20 mM to 30 mM.

Aside from salt, the elution buffer encompasses Bis-Tris Propane and/or Poly(ethylene glycol)-block-poly(propylene glycol)-block-poly(ethylene glycol); also, referred to under the tradenames of Kolliphor® P 188, Poloxamer™ 188 and Lutrol™ F68. The Bis-Tris Propane, when present, is at a concentration of 5 mM, 10 mM, 15 mM, 20 mM, 25 mM, or more. In some embodiments, the elution buffer is a 10 mM. The Poly(ethylene glycol)-block-poly(propylene glycol)-block-poly(ethylene glycol), when present, is at 0.01%, 0.02%, 0.03%, 0.04%, 0.05%, 0.06%, 0.07%, 0.08%, 0.09%, 0.10% or more v/v.

In some embodiments, the elution buffer encompasses a composition encompassing Bis-Tris Propane, Poly(ethylene glycol)-block-poly(propylene glycol)-block-poly(ethylene glycol) and 10-40 mM sodium chloride. In other embodiments, the elution buffer encompasses a composition encompassing Bis-Tris Propane, Poly(ethylene glycol)-block-poly(propylene glycol)-block-poly(ethylene glycol) and 70-80 mM sodium chloride. In still other embodiments, the elution buffer encompasses a composition encompassing Bis-Tris Propane, Poly(ethylene glycol)-block-poly(propylene glycol)-block-poly(ethylene glycol) and 100-120 mM sodium chloride. In some embodiments, the elution buffer encompasses a composition encompassing Bis-Tris Propane, Poly(ethylene glycol)-block-poly(propylene glycol)-block-poly(ethylene glycol) and 40-60 mM sodium chloride. In other embodiments, the elution buffer encompasses a composition encompassing Bis-Tris Propane, Poly(ethylene glycol)-block-poly(propylene glycol)-block-poly(ethylene glycol) and 90-120 mM sodium chloride. In still other embodiments, the elution buffer encompasses a composition encompassing Bis-Tris Propane, Poly(ethylene glycol)-block-poly(propylene glycol)-block-poly(ethylene glycol) and 140-180 mM sodium chloride.

In some embodiments, the elution buffer encompasses a composition encompassing Bis-Tris Propane, Poly(ethylene glycol)-block-poly(propylene glycol)-block-poly(ethylene glycol) and 20 mM sodium chloride. In other embodiments, the elution buffer encompasses a composition encompassing Bis-Tris Propane, Poly(ethylene glycol)-block-poly(propylene glycol)-block-poly(ethylene glycol) and 40 mM sodium chloride. In still other embodiments, the elution buffer encompasses a composition encompassing Bis-Tris Propane, Poly(ethylene glycol)-block-poly(propylene glycol)-block-poly(ethylene glycol) and 80 mM sodium chloride. In some embodiments, the elution buffer encompasses a composition encompassing Bis-Tris Propane, Poly(ethylene glycol)-block-poly(propylene glycol)-block-poly(ethylene glycol) and 100 mM sodium chloride. In some embodiments, the elution buffer encompasses a composition encompassing Bis-Tris Propane, Poly(ethylene glycol)-block-poly(propylene glycol)-block-poly(ethylene glycol) and 120 mM sodium chloride. In other embodiments, the elution buffer encompasses a composition encompassing Bis-Tris Propane, Poly(ethylene glycol)-block-poly(propylene glycol)-block-poly(ethylene glycol) and 140 mM sodium chloride.

In some embodiments, the Bis-Tris Propane is at a concentration of 10 mM and the Poly(ethylene glycol)-block-poly(propylene glycol)-block-poly(ethylene glycol) is 0.01% v/v.

In some instances, the elution buffer for eluting a full capsid differs from the elution buffer for an empty capsid.

### EXAMPLES

### Linear Gradient Scouting Run—Relatively Low Conductivity

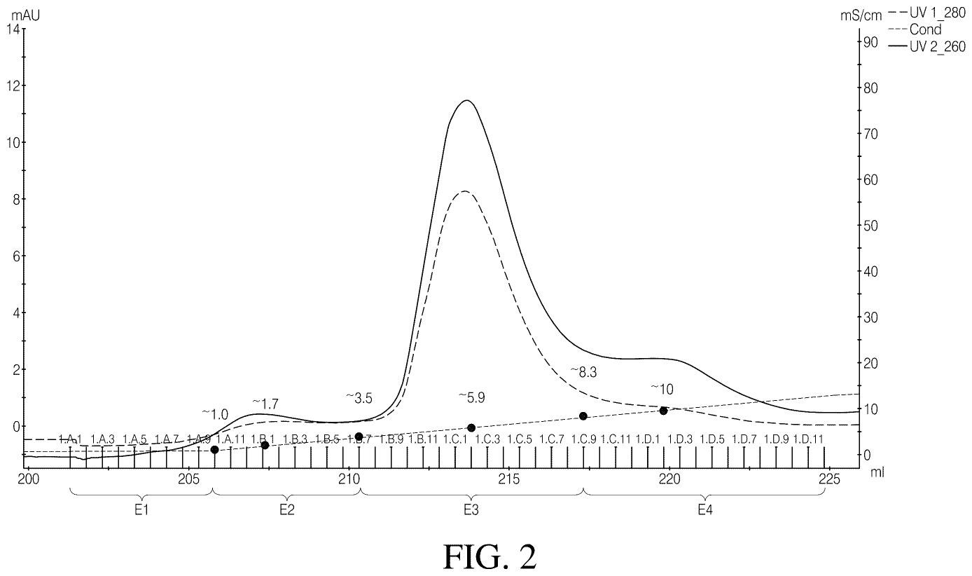

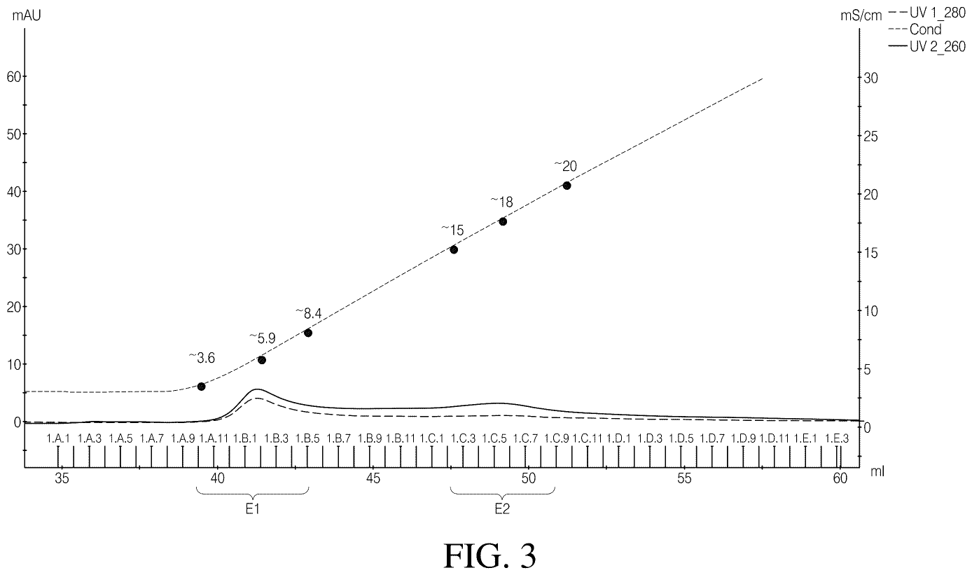

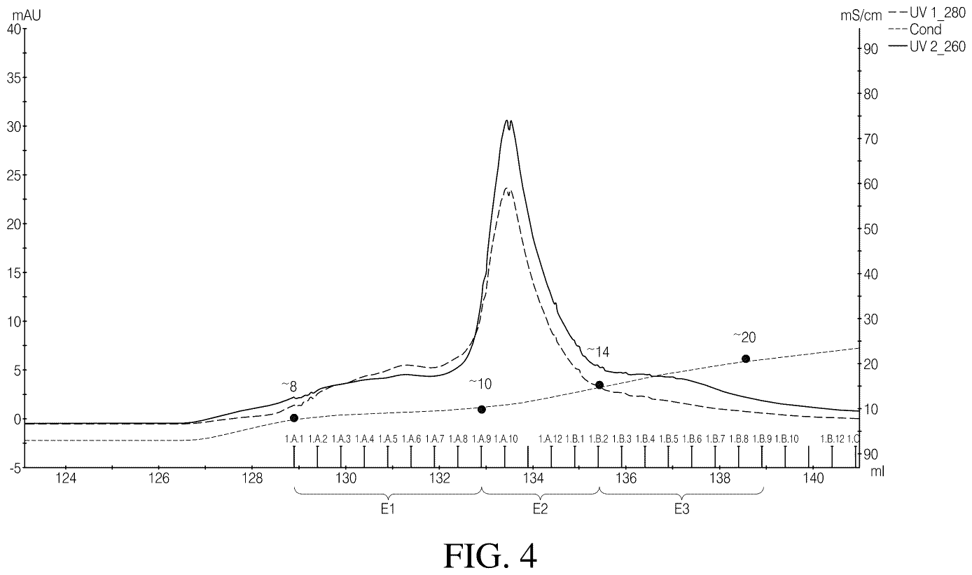

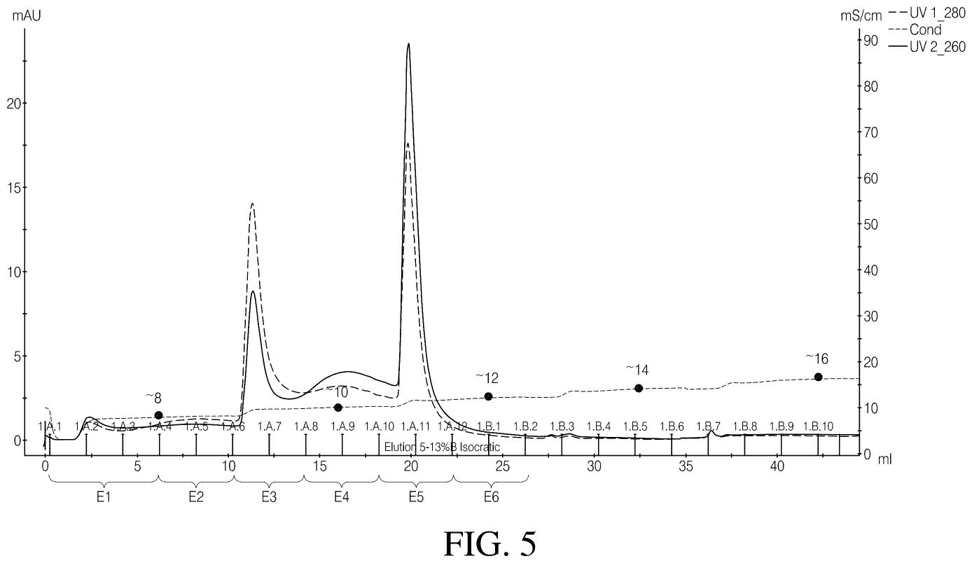

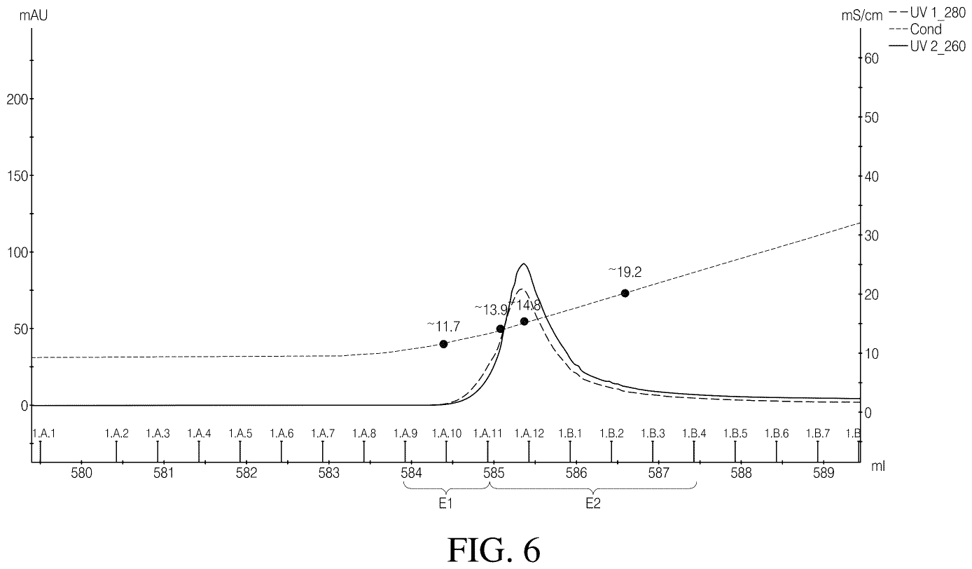

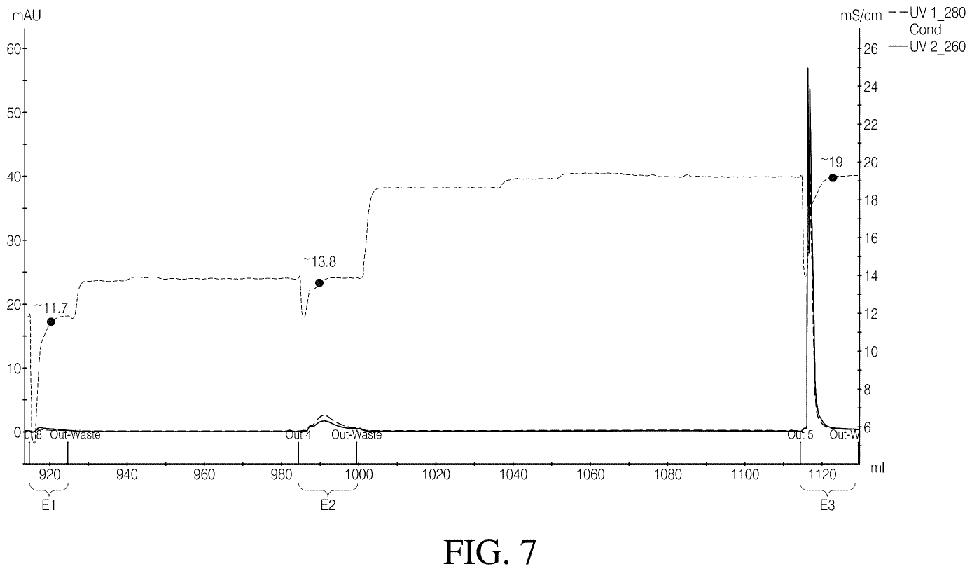

An experimental series was undertaken to identify conditions ultimately establishing compositions allowing for the separation of full from empty AAV capsids without the necessity of an in-line formulation. To these ends, AAV generated from mammalian or insect culture systems was loaded onto a POROS™ CaptureSelect™ AAVX affinity resin. To the AAV containing eluent was added a buffer containing 10 mM Bis-Tris Propane and 0.01% Kolliphor® P 188 (pH 9.0). After addition of this buffer the conductivity of the eluent plus buffer was in the range of 1.5-2.5 mS/cm with a pH>8. This combination of eluent and buffer, referred to as adjusted AAVX eluate or AEX load, was loaded onto a pre-equilibrated anion exchange media. After loading, the anion exchange column was washed with a purification wash buffer containing 10 mM Bis-Tris Propane, 0.01% Kolliphor® P 188 (pH 9.0) and NaCl to remove non-specifically bound material. Bound AAV was then eluted from the washed anion exchange column by increasing ionic strength through a linear gradient. The linear gradient was generated using the mixer on an AKTA chromatography system through the combination of two buffers, the first buffer containing 10 mM Bis-Tris Propane, 0.01% Kolliphor® P 188 (pH 9.0) and NaCl and the second buffer containing 10 mM Bis-Tris Propane, 0.01% Kolliphor® P 188 (pH 9.0) and relatively higher concentration of NaCl. A representative chromatograph from the described anion exchange elution step is show in FIG. 2. Multiple fractions were collected, with collected fraction being no more than twice the volume of the chromatography media. Select fractions were evaluated using vector genome analysis, capsid titer analysis and/or analytical ultracentrifugation in some instances. Evaluated fractions were selected based on chromatograph data, with those fractions identified as containing a peak and those at the start and end of the peak. A peak being any region wherein an increase and decrease in absorbance (UV260 and/or UV280) is observed in the chromatograph. Fractions were also evaluated if they fell within observed UV up or downslopes. Crossover points occur when UV260 and UV280 are of equal values. These can be observed at the start of a peak or at the end of a peak or between two peaks. Fractions neighboring crossover points may have UV280 greater than UV260 values or UV260 values greater than UV260 values.

### Linear Gradient Scouting Run-Relatively Higher Conductivities

Additional scouting runs were undertaken using different buffers with relatively higher concentrations of NaCl and conductivity. These additional scouting runs used experimental conditions largely identical to those applied above with two notable exceptions. First, to the AAV containing eluent was added a buffer containing 10 mM Bis-Tris Propane, 0.01% Kolliphor® P 188 (pH 9.0) and NaCl. This results in the conductivity of the eluent plus buffer being in the range of 3.5-4.5 mS/cm with a pH>8. Secondly, the purification wash buffer contained 10 mM Bis-Tris Propane, 0.01% Kolliphor® P 188 (pH 9.0) and NaCl to remove non-specifically bound material. Other than these modifications, the lower conductivity and higher conductivity linear gradient scouting run protocols were similar but not identical. A representative chromatogram resulting from these conditions is shown in FIGS. 3 and 4. Based on the results from the low and medium adjustment, when undertaken, a decision matrix was generated (Table 2).

TABLE 2

| | AAVX Eluate | Conductivity value at | | |
|---|---|---|---|---|
| | Load | start of first peak/ | Yield in Flow | |
| Scouting | adjustment | region containing | Through (by | |
| study- Load | conductivity | rAAV particles | vector genome | |
| adjustment | (mS/cm) | (mS/cm) | analysis) | Decision |
| Low | 1.5-2.5 | ≥0.3 | <10% | Adjust AAVX Eluate to |
| | | | | a target concentration |
| | | | | of 1.5-2.5 mS/cm |
| Low | 1.5-2.5 | ≥6 | <10% | Adjust AAVX Eluate to |
| | | | | a target concentration |
| | | | | of 1.5-2.5 mS/cm or |
| | | | | compare to medium/ |
| | | | | high load conductivity |
| | | | | experiment results for |
| | | | | desired yield and/or |
| | | | | enrichment. |
| Low | 1.5-2.5 | ≥0.3 | >10% | Additional purification |
| | | | | optimization studies |
| | | | | needed e.g column |
| | | | | type, chemistry, flow |
| | | | | rate, size |
| Medium | 3.5-4.5 | ≥0.5 | <10% | Adjust AAVX Eluate to |
| | | | | a target concentration |
| | | | | of 3.5-4.5 mS/cm |
| Medium | 3.5-4.5 | ≥8 | <10% | Adjust AAVX Eluate to |
| | | | | a target concentration |
| | | | | of 3.5-4.5 mS/cm or |
| | | | | compare to high load |
| | | | | conductivity experiment |
| | | | | for yield and enrichment. |
| Medium | 3.5-4.5 | ≥0.3 | >10% | Conduct low load |
| | | | | adjustment study |
| High | 10.5-11.5 | ≥0.3 | <10% | Adjust AAVX Eluate to |
| | | | | a target concentration |
| | | | | of 10.5-11.5 mS/cm |
| High | 10.5-11.5 | ≥15 | <10% | Evaluate purification with |
| | | | | load adjustment >11.5 mS/cm |
| High | 10.5-11.5 | ≥0.3 | >10% | Conduct low/medium |
| | | | | load adjustment study |

### Performance Optimization by Isocratic Step Gradient

The conditions for enriching full from empty AAV capsids were further refined using an isocratic step gradient based on the linear gradient results (e.g., FIGS. 5 and 7). AAV generated from mammalian or insect culture systems was loaded onto a POROS™ CaptureSelect™ AAVX affinity resin. Based on the results from the linear gradient experiments the appropriate buffer was added to adjust to the desired conductivity. Adjusted AAVX eluate was then loaded onto an anion exchange media. After loading, the anion exchange media is washed with a purification wash buffer. The conductivity of the generated buffer does not exceed that of the adjusted AAVX eluate. Bound AAV was then eluted from the washed anion exchange media by increasing ionic strength using an isocratic step gradient. Elution buffers used, and conductivity ranges, were determined using the results of the linear gradients and with reference to Table 1. Step increases in conductivity were ≥1 mS/cm. The volume of collected fractions was greater than or equal to the column void volume.

Table 3 is representative of yields and fold enrichment of full capsids after an isocratic step gradient scouting experiment (See, also FIGS. 5 and 7).

TABLE 3

| Sample Name | Purification Yield | Enrichment |
|---|---|---|
| E1 + E2 (~8 mS/cm) | 0% | 0.006X |
| E3 + E4 (~10 mS/cm) | 26% | 0.6x |
| E5 + E6 (~12 mS/cm) | 41% | 3x |

Table 4 provides conductivities and salt concentration determination arrived at using linear gradients and applied during isocratic step gradient scouting experiments.

TABLE 4

| | | | | Conductivity | | | |
|---|---|---|---|---|---|---|---|
| | | | | containing | | | |
| Conductivity | Conductivity | Sodium chloride | | enriched | Sodium chloride | | |
| at cross over | at cross over | in buffers | | transgene | in buffers | | |
| #1 (mS/cm) | #2 (mS/cm) | (Table 5) | | particles | (Table 5) | | Serotype |
| ~1 | 3.5 | 10-20 | mM | ~3.5-8.3 | 40-60 | mM | rAAV9.GFP |
| ~8 | ~10 | 70-80 | mM | ~10-14 | 90-120 | mM | rAAV2.GFP |
| ~11.8 | ~13.9 | 100-120 | mM | ~13.9-19.4 | 140-180 | mM | rAAV6.GFP |

Results indicate that enriched transgene containing particles elute at conductivity values of ~12 mS/cm (Table 3). The crossover point using the linear gradient in FIG. 4 is at ~10 mS/cm and the E2 collected after this point also have the highest portion of enriched particles (FIG. 4). Taken together, these results indicated that genome enriched particles can be eluted when the conductivity is ~10-12 mS/cm. A higher portion of transgene lacking particles can be eluted when the conductivity is ~8-10 mS/cm.

With results from the scouting experiments in hand, a table for optimized buffer conditions for isolating column fractions enriched for full AAV was generated (Table 5). For instance, referencing the data presented in FIG. 4, a buffer containing 70 mM and/or 80 mM sodium chloride would eliminate product impurities while a buffer that has 90 mM and/or 100 mM sodium chloride should enrich transgene containing particles.

TABLE 5

| Concentration of sodium chloride | |
|---|---|
| (mM) in 10 mM Bis-Tris Propane, | Conductivity |
| 0.01% Kolliphor pH 9.0 | (mS/cm) |
| 0 | 0.8-0.563 |
| 10 | ~1.67 |
| 20 | 2.73-3.42 |
| 40 | ~5 |
| 50 | 6.3 |
| 60 | ~7.2-7.3 |
| 70 | 8.19-8.27 |
| 80 | 9.23-9.39 |
| 90 | 10.2-10.3 |
| 100 | 11.15-11.42 |
| 120 | 13.12-13.39 |
| 140 | 15.1-15.3 |
| 180 | ~19 |
| 200 | ~21.1 |
| 1000 | 85.9-87.6 |

Isocratic Gradient Enrichment of Full AAV without in-Line Mixing

Based on the results from the linear and isocratic step gradients scouting experiments, conditions for off-line isocratic enrichment of full AAV were undertaken. An overview of the off-line isocratic conditions applied are provided in Table 1. The enrichment of full capsids of various serotypes is shown in Table 6.

TABLE 6

| | | Purification | |
|---|---|---|---|
| Serotype | Fraction | yield (%) | Enrichment |
| rAAV2 | Wash #1 (70 mM) | * | * |
| | Elution #1 (80 mM) | 0 | ~0.007 |
| | Elution #2 (100 mM) | 55% | ~2 |
| rAAV9 | Wash #1 (10 mM) | * | * |
| | Elution #1 (20 mM) | * | * |
| | Elution #2 (40 mM) | 72% | ~2.5 |
| rAAV6 | Wash #1 (100 mM) | * | * |
| | Elution #1 (120 mM) | * | * |
| | Elution #2 (140 mM) | 66% | ~1.7 |

1. A method for enriching for full AAV capsids, the method comprising loading a sample suspected of comprising empty and full AAV capsids onto an anion exchange chromatography medium, differentially eluting the full AAV capsids and the empty AAV capsids with an elution buffer, the composition of the elution buffer is not prepared in-line.

2. The method of claim 1, wherein the sample is a cell lysate or cell culture supernatant.

3. The method of claim 2, wherein the cell lysate or supernatant is derived from a mammalian cell or an insect cell.

4. The method of claim 1, wherein the sample is clarified before loading onto the anion exchange chromatography medium.

5. The method of claim 1, wherein the sample is derived from a culture medium of 10 liters or more.

6. The method of claim 1, wherein the sample is derived from a culture medium of 50 liters or more.

7. The method of claim 1, wherein the sample is an affinity column eluent.

8. The method of claim 7, wherein the affinity column binds AAV.

9. The method of claim 1, wherein the sample is clarified before loading onto the affinity column.

10. The method of claim 1, wherein the AAV is a serotype selected from a naturally or non-naturally occurring serotype.

11. The method of claim 10, wherein the serotype is selected from 1-9, rh10, 11, or 12.

12. The method of claim 1, wherein the anion exchange chromatography medium comprises a monolithic column.

13. The method of claim 1, wherein the elution buffer comprises Bis-Tris propane.

14. The method of claim 1, wherein the elution buffer comprises Poly(ethylene glycol)-block-Poly(ethylene glycol)-block-Poly(ethylene glycol).

15. The method of claim 1, wherein the elution buffer comprises a monovalent salt or a divalent salt.

16. The method of claim 1, wherein the elution buffer comprises both a monovalent and a divalent salt.

17. The method of claim 1, wherein the elution buffer comprises a monovalent salt.

18. The method of claim 17, wherein the monovalent salt is NaCl.

19. The method of claim 18, wherein the NaCl concentration is between 1 mM and 1000 mM.

20. The method of claim 18, wherein the NaCl concentration is between 1 mM and 200 mM.

21. The method of claim 18, wherein the NaCl concentration is between 1 mM and 140 mM.

22. The composition of the elution buffer used in claim 1.

23. A method for identifying the elution buffer composition of claim 1, the composition arrived at through the combination of results from linear gradient and step isocratic anion exchange chromatography, the formulation of the linear gradient elution buffer being prepared in-line.

24. A full AAV capsid isolated using the method of claim 1.
